(19) United States
(12) Patent Application Publication
(10) Pub. No.: US 2021/0005321 A1
(43) Pub. Date: Jan. 7, 2021

(54) SYSTEM AND METHOD FOR PREDICTING PATIENT RISK OUTCOMES

(71) Applicant: DEPUY SYNTHES PRODUCTS, INC., Raynham, MA (US)

(72) Inventor: Julia HWANG, Wayland, MA (US)

(21) Appl. No.: 16/585,927

(22) Filed: Sep. 27, 2019

Related U.S. Application Data
(60) Provisional application No. 62/870,329, filed on Jul. 3, 2019.

(51) Int. Cl. G16H 50/30; G16H 50/20; G16H 10/60

(52) U.S. Cl. CPC G16H 50/30; G16H 10/60; G16H 50/20

(57) ABSTRACT

A predictive modeling engine to compute risk outcomes for a patient being considered for a medical procedure. Information indicative of the risk outcomes, including risk scores, are output on graphical user interface of an interactive display. The risk outcomes may be displayed with various types of information that may assist doctors, surgeons, and other healthcare professionals to make decisions on whether the procedure should be performed on the patient, given the computed risk outcomes. The predictive modeling engine may be implemented by one or more machine-learning algorithms, that may include linear regression and/or other types of processing.

This application is based upon and claims the benefit of priority from prior Provisional Application No. 62/870,329, filed Jul. 3, 2019, which is hereby incorporated by reference for all purposes as if fully set forth herein.

### TECHNICAL FIELD

Example embodiments disclosed herein relate generally to processing medical information, and more specifically to a system and method for predicting patient risk outcomes.

### BACKGROUND

The difficulty of making medical decisions has continued to get more complicated over time. Healthcare professionals generally rely on their experience in deciding whether to administer certain courses of treatment or perform specific types of surgeries. However, the risks to one patient may be different from the risks to another patient for the very same procedure. In many cases, doctors are not even aware of all the risks that may be involved. As a result, quality care may diminish or a more effective procedure may have been available that would be less burdensome to the patient.

Currently, there exists no processing tools that may be relied on by doctors, surgeons, and other care agents that can determine the risks associated with performing medical procedures for patients with different medical conditions.

### SUMMARY

One or more embodiments include a system for processing information that includes an input configured to receive data relating to a patient through a network; a display controller configured to control output of information on a display; and a predictive modeling engine configured to compute one or more risk outcomes based on the patient data, the one or more risk outcomes computed for a procedure to be performed on the patient, each of the one or more risk outcomes including a risk score for the patient, wherein the display controller is configured to control display of information indicative of the risk scores and one or more modifiable factors, the predictive modeling engine configured to change the risk scores based on changes in the one or more modifiable factors.

### BRIEF DESCRIPTION OF THE DRAWINGS

Additional objects and features of the invention will be more readily apparent from the following detailed description and appended claims when taken in conjunction with the drawings. Although several example embodiments are described, like reference numerals identify like parts in each of the figures, in which:

FIG. 1 illustrates an embodiment of a system for predicting risk outcomes associated with a medical procedures that may be performed on a patient;

FIG. 2 illustrates an example of input information and risk outcomes for the system of FIG. 1;

FIG. 3 illustrates an example of a screen generated by a graphical user interface according to one embodiment;

FIG. 4 illustrates an example of another screen generated by the graphical user interface according to one embodiment;

FIG. 5 illustrates an example of a logistic regression ROC curve for a predictive model generated for a readmission outcome for a total knee arthroplasty (TKA);

FIG. 6 illustrates an example of another screen generated by the graphical user interface according to one embodiment;

FIG. 7 illustrates an example results from the predictive model for hospitals;

FIG. 8 illustrates an embodiment of a method for calculating risk scores associated with one or more of risk outcomes.

FIG. 9 illustrates an example graph of readmission risk scores for a sample population of patients;

FIG. 10 illustrates an example of a graph where the rate of incidence is calculated from probability predictions from the model for Total Knee Replacement surgery.

FIGS. 11A to 11J illustrate examples of additional screens generated by the graphical user interface according to one or more embodiments;

FIG. 12 illustrates an embodiment of a method for predicting risk outcomes associated with medical procedures that may be performed on a patient; and

FIG. 13 illustrates an embodiment of a processing system in accordance with one embodiment.

### DETAILED DESCRIPTION

The descriptions and drawings illustrate the principles of various example embodiments. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various example embodiments described herein are not necessarily mutually exclusive, as some example embodiments can be combined with one or more other example embodiments to form new example embodiments. Descriptors such as “first,” “second,” “third,” etc., are not meant to limit the order of elements discussed, are used to distinguish one element from the next, and are generally interchangeable. Values such as maximum or minimum may be predetermined and set to different values based on the application.

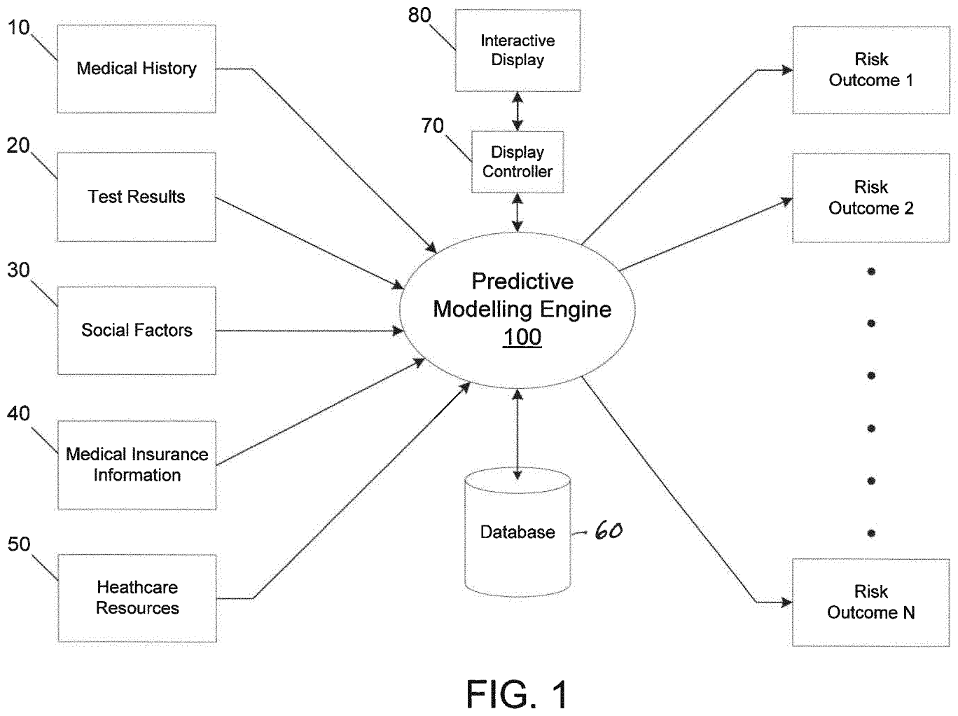

FIG. 1 illustrates a system for predicting patient risk outcomes for various types of medical procedures (e.g., surgeries, treatments, etc.). The system may be used, for example, by healthcare organizations and professionals in determining the most appropriate courses of action given the particular conditions and circumstances of each patient.

Referring to FIG. 1, the system includes a predictive modeling engine 100 that computes one or more predictive risk outcomes based on a number of inputs. The inputs may include medical history information 10, test results 20, social factors 30, medical insurance information 40, and healthcare resources 50. This information may be stored, for example, in one or more databases in the form of electronic medical records. The databases may be located within a medical facility treating a patient and/or may be remotely located from the medical facility. The information stored in the databases may be accessed, for example, through one or more networks coupled to a processing system which includes the predictive modeling engine 100. The networks may be various types of local or wide area networks that transmit encrypted data for protecting the privacy interests of patients and healthcare organizations and professionals.

The medical history information 10 is different for each patient and may indicate, for example, previous diagnoses, procedures, treatments, and healthcare usage. The information may be stored in the form of electronic medical records obtained from doctor offices, clinics, hospitals, and other medical and healthcare-related sources. The medical history information may also include a listing of medications patients have taken and are currently taking. All of this information may be used to predict the risk(s) associated with various healthcare treatment that may be under consideration for a patient.

The test results 20 may include laboratory values produced from blood tests, liver function tests, kidney function tests, thyroid function tests, pulmonary function tests, electrolyte analyses, bone density studies, prostate specific antigen tests, malabsorption tests, gastric fluid analyses, pap smears, pregnancy tests, and urinalysis, as well as other tests. The test results information may also include results obtained from various procedures, including but not limited to colonoscopies, x-rays, computed tomography scans, magnetic resonance scans, mammograms, electrocardiogram and other heart-related tests, and biopsies, as well as other types of medical information. The test information may also include weight and height as well as patient vital signals including blood pressure, pulse, temperature, oximeter readings, and other information obtained from monitoring equipment.

The social factors 30 may include information indicating whether the patient is a smoker or drug user, whether and how much the patient drinks, lifestyle information, marital status, country of origin, ethnicity, race, house hold income, education level, distance from health care system, recent travel information, and other information that might serve as a basis for determining whether the patient is subject to a particular risk for a given medical procedure under consideration.

The medical insurance information 40 may include types of medical insurance coverage, prescription information, payment plans, claims history, pre-existing conditions, Medicare information, pre-authorizations, qualifications, and other types of information relating to the medical insurance of a patient under consideration.

The healthcare resources 50 may include availability and scheduling information of surgical resources and personnel in a medical facility, intra-operative variables, capabilities and equipment for performing surgical and other types of medical procedures. This information may include, for example, any specialties or other areas of expertise at the facility (e.g., shock-trauma center, burn units, cancer specialists, behavioral wards, etc.) that may have a bearing on a potential risk outcome.

The predictive modeling engine 100 may use one or more algorithms to compute risk outcomes for each patient under consideration. The risk outcomes may be computed, for example, for possible treatment options. For example, consider the case where a doctor is considering whether a patient should undergo a particular surgery to treat a condition. The predictive modeling engine 100 may compute one or more risk outcomes 1 to N for the patient if the surgery is performed. The outcomes may include, for example, the risk of the patient surviving the surgery, the risk that the surgery will be unsuccessful, the risk that infection or other complications will develop, and the risk that other conditions of the patient may be adversely affected. The risk outcomes may also include more specific risks, such as risks associated with readmission to the hospital, developing an infection, increased length of stay, cardiac complications, renal complications, respiratory complications, re-operation (revision), re-operation (non-revision), and as well as prediction on discharge location. These and/or other risk outcomes are computed based on the information input into the predictive modeling engine 100.

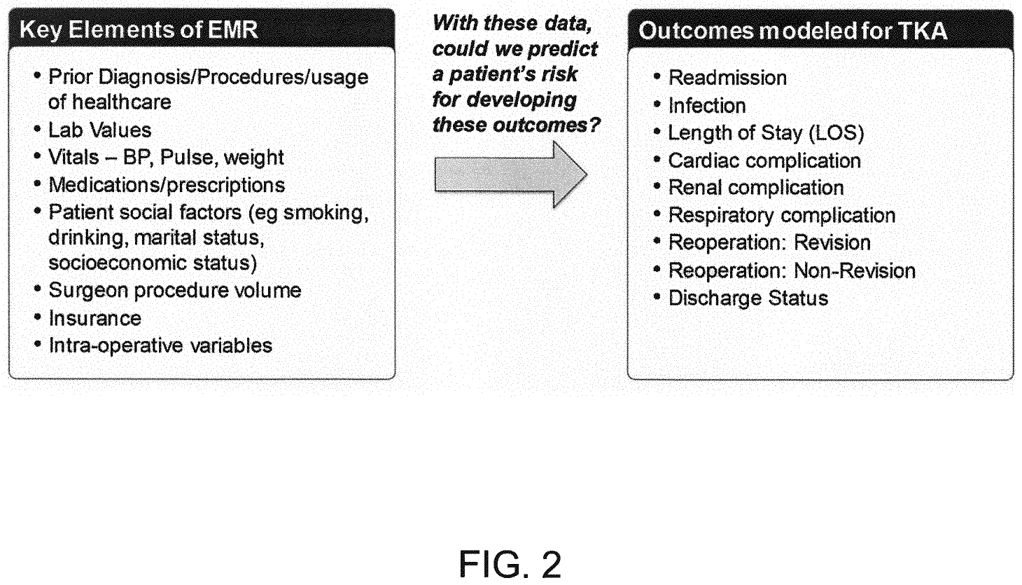

FIG. 2 illustrates a conceptual diagram showing examples of input information and risk outcomes that may be generated in accordance with one or more embodiments. The input information (listed under the column entitled “Key Elements of EMR”) corresponds to many of the types of input information previously described. The risk outcomes may correspond to a static list of risk outcomes that are considered irrespective of the patient and the prevailing condition or may correspond to a dynamic list of risk outcomes generated by the predictive modeling engine 100 given the input patient information. In this latter case, the list of risk outcomes may be different for each patient, thus customizing the risks on a patient-by-patient basis.

In one embodiment, the predictive modeling engine 100 may generate a score or some other quantitative or qualitative measure of risk associated with each outcome. The score may be generated, for example, relative to a predetermined benchmark (e.g., an average or some other statistical measure) and/or relative to the risks associated with other generated outcomes. The scores may be displayed in a uniquely generated graphical user interface that, for example, may combine various sources and type of information on one or more screens with the risk outcomes. Such an interface may allow a doctor or other healthcare professional to make an efficient determination of whether to go forward with a certain procedure or treatment given the associated risks. The predictive modeling engine 100 may therefore serve as a tool for providing guidance in making optimal healthcare decisions for patients.

The predictive modeling engine 100 may use various algorithms to generate the risk outcomes for each patient. The algorithms include machine-learning algorithms, e.g., random forest algorithm, extreme gradient boost algorithm, naïve Bayes algorithm, K-nearest neighbor algorithm, support vector machines, neural networks, and logistic regression models, to name a few. The algorithms may be used individually or may be considered in combination when computing the final risk outcomes. As illustrated in FIG. 1, the risk outcomes may be output in one or more predetermined formats and/or screens of an interactive display 80 using a display controller 70.

The display controller 70 and interactive display 80 may be on a local computer storing the algorithms for implementing the predictive modeling engine 100. In another embodiment, the display controller 70 and interactive display 80 may be in a remote device that communicates with a server or other computer through a wired or wireless network connection. The remote device may be a smartphone, tablet, laptop or notebook computer, or another type of mobile device.

In one embodiment, the algorithm(s) used and the risk outcomes generated by the predictive modeling engine 100 may be unique, specific, or different for a given medical facility (e.g., hospital, out-patient surgery center, clinic, etc.) and/or geographical region. For example, risk outcomes may be different given different environmental conditions that exist in different regions of the country or world, different areas of expertise, specialists, or equipment, or programs available, different patient demographics, and/or based on other differences.

The predictive modeling engine 100 may be trained, for example, based on electronic medical record (EMR) data, as well as a test set of patient data corresponding, for example, to the relevant geographical area and/or patient population. In one embodiment, the algorithm(s) used by the predictive modeling engine may be derived from mobile devices, wearable devices, and/or monitoring equipment or other tools used in the home, operating room, or hospital. Training the algorithm(s) of the predictive modeling engine may also involve engineering the training data, for example, by setting or changing categorical variables to numeral variables. The levels of data may also be concentrated (e.g., reduced) to focus on only a predetermined subset of information, e.g., marital status may be reduced from 7 categories to 2 categories. Additionally, various comorbidity indexes (e.g., Elixhauser, Charlson, Functional, etc.) may be specially designed from diagnosis codes and used to train the predictive modeling engine. In one embodiment, in training the model, only the most important features may be selected for each data element. The selection may be performed, for example, based on recursive feature elimination with a cross-validation technique. Because at least some the risk outcomes to be predicted may constitute a rare occurrence (e.g., misbalanced data), oversampling and under-sampling techniques may also be used for training purposes. For example, in one application a hybrid or up and down sampling approach with bootstrapping may be used to reduce variance.

In one embodiment, the predictive model may be trained based on a dataset of N observations, where N may be at least a predetermined number of observations. For example, for a total knee arthroplasty (TKA) example, the value of N may be 24,000 or more. In other embodiments, N may be less than or greater that this number. In some cases (not just a TKA procedure), there may be significant class imbalance in the dataset used to train the mode. This problem may occur, for example, in medical diagnosis datasets. In order to compensate for class imbalance, a hybrid up/down sampling technique (bootstrapping) may be used. For example, randomly down/up (bootstrapping) sample training sets, with replacement, may be used as a basis for generating a test set. The test set may then be used to generate prediction outcomes toward generating a valid set of data, for example, according to the following equation:

$$\frac{\sum probability}{number of bootstraps} ( \% )$$

In some cases, techniques which involve under-sampling the majority and over-sampling the minority of observations in the dataset may be performed to train the predictive model.

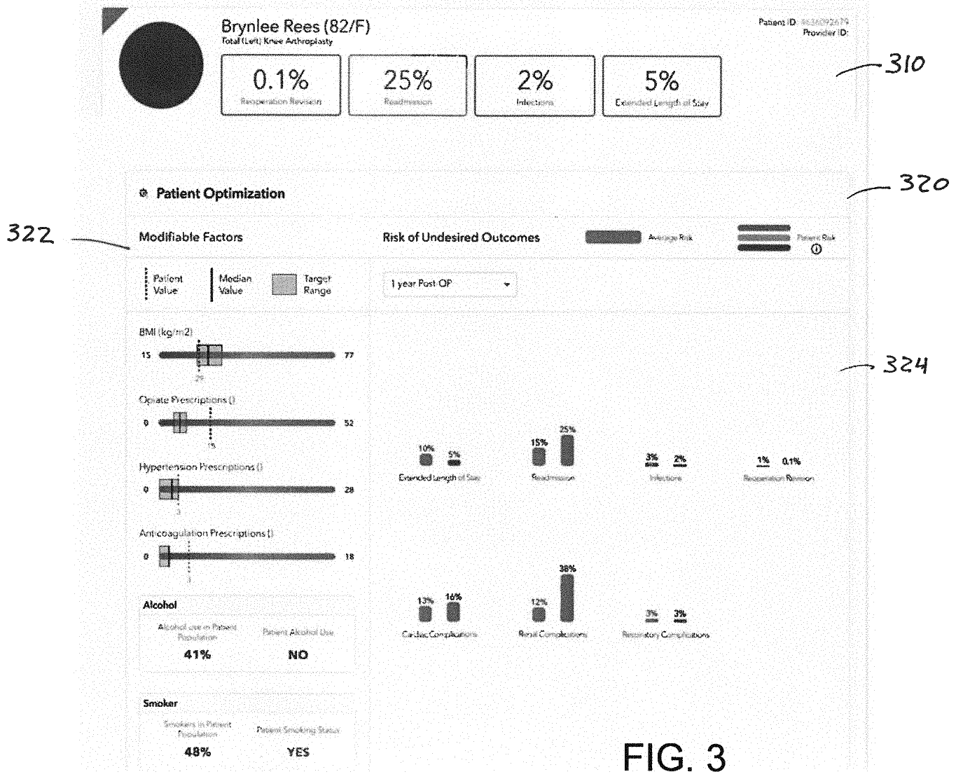

FIG. 3 illustrates a screen that may be displayed on a graphical user interface in accordance with one embodiment. This screen may indicate risk outcomes generated by the predictive modeling engine 100 for a given patient. In this example, the screen may include a first section 310 for identifying a patient and a second section for displaying the risk outcomes for that patient.

The first section 310 may include, for example, the name and an image of the patient along with one or more types of demographic information (e.g., sex, age, etc.) and an indication of the treatment that is associated with the risk outcomes. In this example, doctors or other healthcare professionals are considering the risks involves with performing a left knee arthroplasty on an 82 year-old, female patient named “Brynlee Rees.”

In addition to these features, the first section 310 may indicate all or a portion of the risk outcomes generated by the predictive modeling engine 100, with an indication of their risk scores. In this example, there is 0.1% chance that patient Brynlee Rees will have to undergo a re-operation revision if the arthroplasty procedure is performed. The first section 310 also indicates that there is a 25% chance of readmission, a 2% chance of infection, and a 5% chance that an extended length of stay will be required if the arthroplasty procedure is performed on Brynlee Rees. The specific type of risk outcomes may be specifically set (e.g., based on a user input) to be shown in the first section, or only a predetermined number of the highest risk outcomes may be displayed in this section. As previously indicated, these risk outcomes are generated based on input information specifically relating to this patient (as indicated, for example, in FIG. 1) and/or the healthcare resources that are available.

The second section 320 allows a user to optimize the risk outcomes computed by the predictive risk engine. For example, the second section may include a first area 322 indicating a number of modifiable factors that may allow a user to set or change values corresponding to one or more inputs used to generate the risk outcomes. In this example, the modifiable factors include body mass index (BMI), opiate prescription(s), hypertension prescription(s), and anticoagulation prescription(s). The patient value is shown as dotted lines and the value of an average population is shown as a box with median values denoted as a solid line within the box. The average range may be treated as a target range for the patient. Values for patient factors may be modified (e.g., by adjusting sliding cursors) or it may be a static representation showing the patient value relative to an average population undergoing the associated procedure. Patient values may be adjusted to lie within or outside of the average range window, to produce different risk outcomes in the judgment of the physician or other healthcare professional. In addition to these features, the second section 320 may also include information corresponding to one or more social factors. In this example, the social factors information indicates that patient Brynlee Rees does drink alcohol (but 41% in the relevant patient population drinks alcohol) and is a smoker (and that 48% of the population smoke).

Setting or changing the modifiable factors in area 322 causes the predictive modeling engine 200 to generate corresponding risk outcomes that are set or changed in another area 324 of the second section of the graphical user interface. In this example, area 324 shows seven possible risk outcomes under consideration, namely extended length of stay, readmission, infections, re-operation revision, cardiac complications, renal complications, and respiratory complications. Each risk outcome is displayed with an associated risk score specific to the patient under consideration (e.g., Brynlee Rees). In one embodiment, the patient score for each outcome may be displayed in association with an average risk score, for example, given the relevant patient population for the procedure, e.g., arthroplasty in this example. The average risk score may be defined as all patients for a given hospital or doctor or region undergoing relevant procedure. The patient optimization section 320 may also include a menu to allow a user to select other parameters for the risk outcomes. In the example shown, one-year post-operative is selected as the parameters for the risk outcomes displayed in section 324.

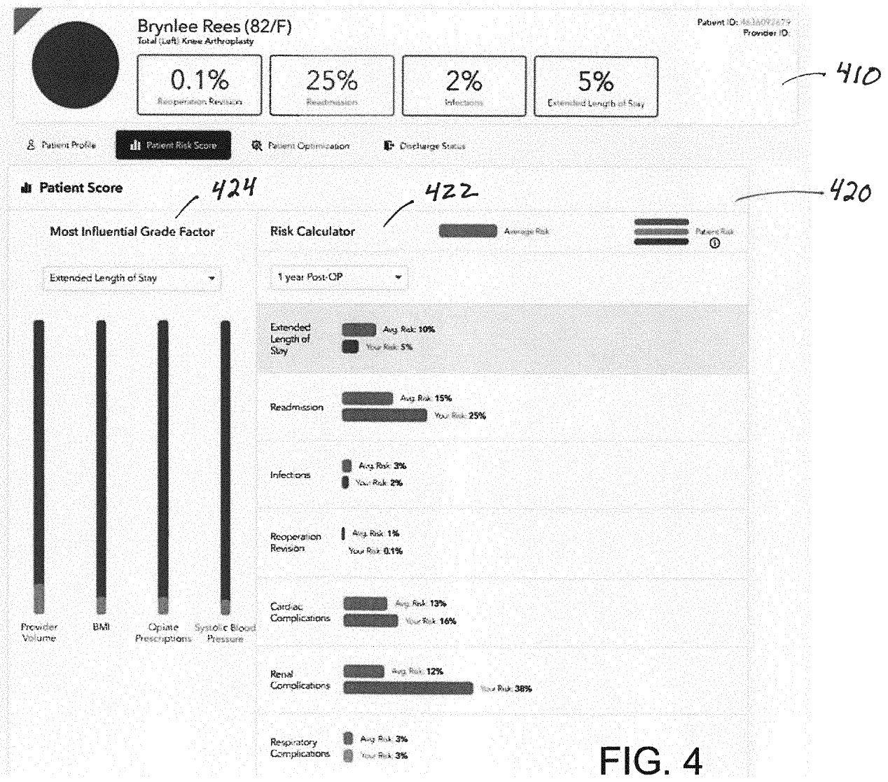

FIG. 4 illustrates an example of a screen that may be displayed on the graphical user interface to provide a summary of patient risk factors that may help surgeons (or other healthcare professionals) make decisions on whether a patient should undergo a certain procedure (e.g., treatment, surgery, etc.). The screen in FIG. 4 may include a first section 410 and a second section 420. The first section may include the same information as section 310 in FIG. 3, except that the risk outcome that was selected may be distinguished (e.g., by color or other graphical indication) from the other risk outcomes.

The second section 420 may include a patient score section having a first area 422 and a second area 424. The first area 422 may correspond to a risk calculator which shows the risk scores for each of the risk outcomes in FIG. 3. The risk score generated by the predictive modeling engine 100 may be displayed in association with an average risk score for a relevant patient population, geographical area, medical facility, etc. In one embodiment, the population may be chosen based on a preference of the user, e.g., age, matched cohort, all patients of a particular surgeon, all hospital patients, etc.

In one embodiment, each risk outcome in the first area 422 may be selectable. Selecting a risk outcome in first area 422 determines the information displayed in the second area 424. Instead of selecting risk outcomes in the first area 422, in one embodiment the second area 424 may include a drop-down menu listing the risk outcomes. Selecting one of the risk outcomes in the drop-down menu may cause most influential grade factors for that outcome to be displayed. The information corresponding to the risk factor in the first area 422 may be highlighted based on the selection.

The second area 424 may display the most influential grade factors computed by the predictive modeling engine 100 for the risk outcome selected in first area 422. For example, when the risk outcome corresponding to extended length of stay is selected in the first area, the one or more (in this example, four of the) most influential grade factors determined by the predictive modeling engine 100 are displayed in second area 424 for patient Brynlee Rees for the arthroplasty under consideration. In this example, the most influential grade factors for the risk outcome corresponding to extended length of stay include provider volume (e.g., number of relevant procedures performed by the health care professional), BMI, opiate prescriptions, and systolic blood pressure. The height of the bars denote the relative important of that feature to the model. The higher the value for the bar, the more that feature impacts the risk outcome. The values for these factors may be graphically indicated, for example, by displaying a highlighted bar. When other risk outcomes are selected in the first area 422, the same or different most influential grade factors may be displayed in the second area 424, as determined by the predictive modeling engine.

Thus, the screen in FIG. 4, provides a summary of patient risk factors that may help surgeons (or other healthcare professionals) to make a decision on suitability of the patient for surgery (or other treatment) that is under consideration. In one embodiment, the present system may be used as a tool prior to surgery, after surgery/prior to discharge, or at other times for purposes of helping professionals made patient care decisions, in view of impending risk outcomes. Updates to the risk calculator may be performed by the predictive modeling engine when new or modified input information is available and downloaded to the system for use by the predictive modeling engine. The input information and/or the information corresponding to the computed risk outcomes may be stored, for example, in the database 60 of FIG. 1.

In one embodiment, the predictive modeling engine 100 may calculate the data in the screen of FIG. 4 for a predetermined number of time points, e.g., a first timepoint at 90 days and a second timepoint of 1 year. Data for different and/or additional timepoints (e.g., a 30-day timepoint) may calculated and displayed in other embodiments. Instead of selecting risk outcomes in the first area 422, the second area 424 may include a drop-down menu listing the risk outcomes. Selecting one of the risk outcomes in the drop-down menu may cause most influential grade factors for that outcome to be displayed. The information corresponding to the risk factor in the first area 422 may be highlighted based on the selection.

In one embodiment, the importance scores may be generated for the most influential grade factors relating to the outcome of readmission given the type of surgery to be performed. The most important features may be determined, for example, based on a recursive features elimination with cross validation (RFECV) technique. RFECV may fit to the model and the weakest feature(s) may be removed until a specified number of features is reached. The features may then be ranked by coefficients of the model (or feature importances), as well as by recursively eliminating a predetermined (e.g., relative small) number of features per loop. The recursive feature elimination may be applied in a manner which attempts to eliminate dependencies and collinearity in the model, if any. Cross-validation may then be used to determine the optimal number of features, and may be combined with recursive features elimination to score different features subsets and to select the best scoring collection of features.

Examples of outcomes involving readmission include, but are not limited to, infection, cardiac complications, renal complications, and respiratory complications. Separate predictive models may be generated for type of outcome based on associated datasets, as described in accordance with the embodiments herein. Additional factors taken into consideration for determining risk of readmission using the predictive model include arthrofibrosis, aseptic loosening, patellofemoral dislocation, reoperation: revision, and reoperation: non-revision.

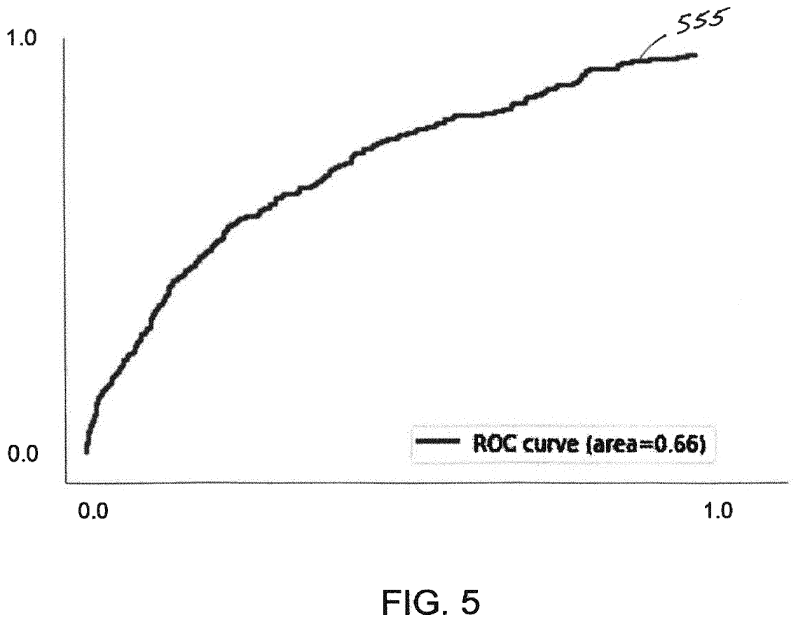

FIG. 5 illustrates an example of a logistic regression ROC curve 555 corresponding to a predictive model generated based on datasets for the TKA example. The accuracy of the risk scores generated using such a predictive model shows a rate of 66.47% for the outcome of readmission over a period of three months from the TKA surgery. The area under the bagged logistic regression for the same was 65.80%. Examples of these results are shown in the table below. In generating these numbers, the predictive model generated 1991 true positives (no complication), 110 false positives (false prediction of no complication), 994 false negatives (false prediction of complication), and 204 true negatives (true complication). The sensitivity=1991/(1991+994)=67% and the specificity=204/(110+204)=65%.

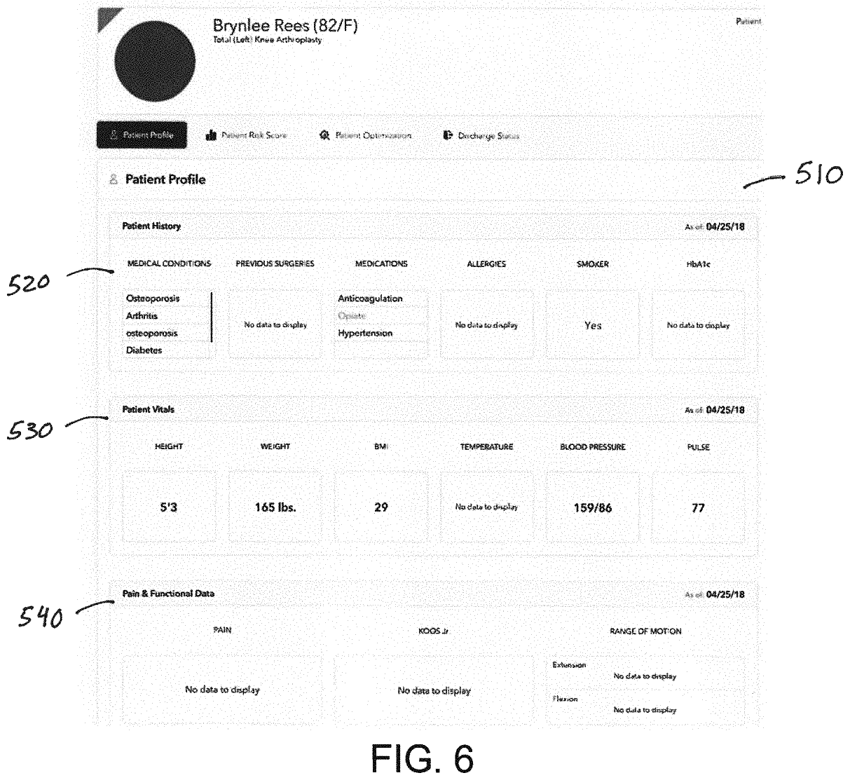

FIG. 6 illustrates another screen that may be displayed on the graphical user interface. This screen includes a patient profile section 510 indicating various data relating to patient Brynlee Rees. For example, the patient profile section may include a patient history section 520, a patient vitals section 530, and a pair and functional data section 540. The information in these sections may be combined within a single screen that may be reviewed, for example, by a surgeon and/or his staff for purposes of reviewing the condition of the patient. The data in one or more of these sections may be based, for example, on electronic medical records obtained from databases connected to a network.

The patient history section 520 may output data corresponding to the medical history and other information input into the predictive modeling engine 100 of FIG. 1. In the case of patient Brynlee Rees, the patient history section 520 indicates medical conditions of Ms. Rees, previous surgeries, medications, and allergies, as well as other health-related information. The patient history section may also indicate social factors such as whether the patient is a smoker, an alcohol drinker, or a recreational drug user.

The patient vitals section 530 indicates height, weight, body mass index (BMI), temperature, blood pressure, and pulse, as well as other vital sign information that may be considered relevant given the procedure to be performed or which otherwise may be indicative of the general health status of the patient.

The pair and functional data section 540 may include information indicating current or last-reported pain levels of the patient, KOOS JR. information, and range of motion. The information in section 540 may differ based on patient condition and/or the procedure (e.g., treatment, surgery, etc.) associated with the risk outcomes generated by the predictive modeling engine. In one embodiment, the information displayed in sections 520, 530, and 540 may include a time and/or date stamp to show when the information was captured or otherwise pertained to the patient. A user may be allowed to highlight, flag, or otherwise emphasize certain information or factors that might be especially relevant to the patient condition and/or the procedure to be performed.

The risk scores generated by the predictive models generated in accordance with the embodiments described herein may be performed for different medical facilities, e.g., different hospitals, for comparative purposes. Under these circumstances, each model may be trained based on datasets that are limited to conditions and patients relate to one hospital only.

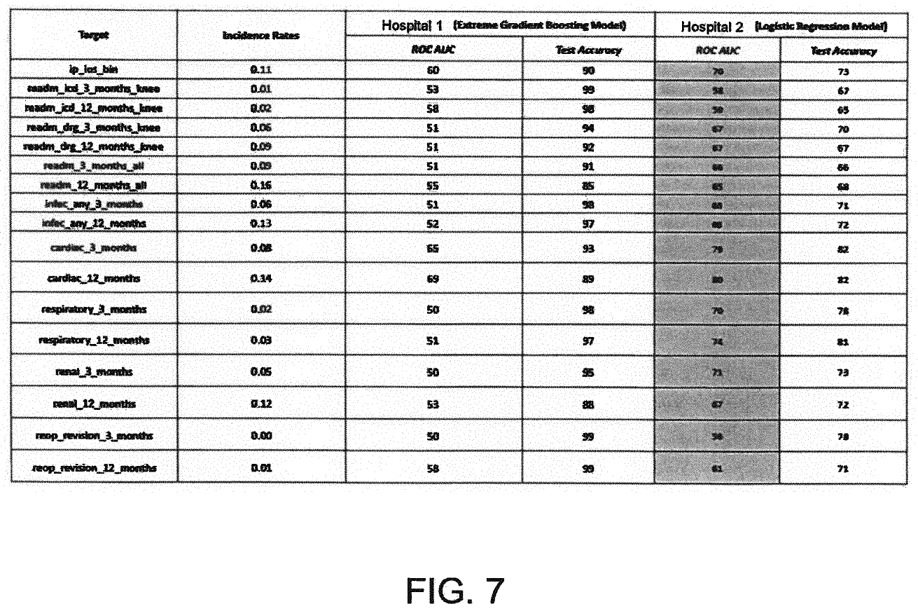

The risk scores generated by the predictive models may therefore provide indications, especially for readmission, of corresponding probabilities of complications that may occur on a procedure-by-procedure basis. The risk scores for readmission may vary from hospital to hospital based on varying conditions and data on which the models for the hospitals are trained. This is evident from the example data set forth in the chart of FIG. 7, which compares data and scores for readmission associated with two hospitals (e.g., Hospital 1 and Hospital 2) relating to a TKA procedure.

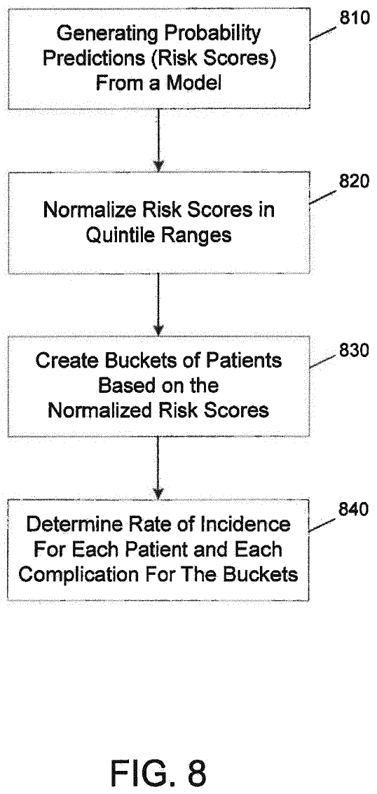

FIG. 8 illustrates an embodiment of a method for calculating risk scores associated with one or more of the risk outcomes discussed herein. For illustrative purposes, the risk outcome associated with this method embodiment will be discussed as corresponding to readmission, and more specifically to the probability of complications occurring after an initial episode with a medical facility that likely result in readmission of patients. The medical facility may be a hospital, clinic, outpatient care center, or another type of facility. In some cases, the predictions generated by a model may not accurately reflect the rate of complications that actually occur. The method of FIG. 8 may be implemented to improve the accuracy of the predictions relative to complications that may likely occur for a given procedure. This may involve transforming the model predictions to risk scores that represent a more accurate probability that there will be a complication that results in readmission for a given procedure.

At 810, the method includes generating probability predictions from a model. In the example under consideration, the probability predictions correspond to risks of readmission calculated based on samples of patient data. For example, before the probability predictions are generated, the patient data may be pre-processed and then used to train the model. Pre-processing may include identifying independent and dependent variables. The variables may be identified, for example, by surgeons and may change over time.

Then univariate analysis and outlier treatment may be performed. This may involve deriving basic statistics of the patient data, central tendency, spread and missing values based on the identified variables, e.g., based on a distribution of linear variables for all patients. Probability density plots and box plots may be created for the variables. Outlier treatment may also be performed so that the results are not skewed and are more reliable.

Then, a correlation matrix may be generated with key variables to evaluate potential correlations between or among variables. This may be performed, for example, by creating scatter plots and/or trellis plots where appropriate. This may be followed by an operation of removing correlated and colinear variables, which may be performed based on a collinearity analysis. Recursive Feature Elimination may then be performed for dimensionality reduction before passing the data to the model for training. The readmission risks (or probabilities) generated by the model may be expressed, for example, as scores as previously discussed. The risk scores may lie within a range of 1 to 100, with a score of 1 being the lowest possible risk and 100 the highest possible risk.

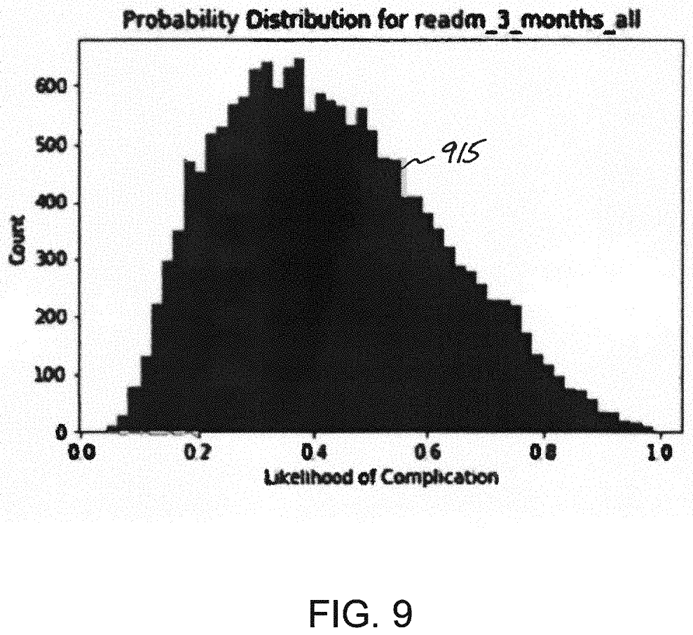

FIG. 9 illustrates an example graph of readmission risk scores 915 for a sample population of patients. The graph indicates a probability distribution for readmission within three months for the example of knee replacement surgery. The mean of the probability distribution is centered around 40% to 50% for the outcomes under consideration, and the rate of complication is as low as 3%. At this point, it is possible for the probability distribution is not the best way to represent outcomes that have low complication rates. For example, a person at the 100^{th} percentile may not really have a 100% chance or readmission. In reality, the actual incidence of this outcome may be much lower than 100%. Similarly, a patient in the 50^{th} percentile may not really have a 50% chance of readmission. Again, the rate of incidence may be lower. In order to show the user more relevant values, the probability values need to be normalized.

At 820, the risk scores in the probability distribution may be normalized to an incidence rate. For example, the normalization may be performed for each quintile in the distribution. For patients that fall into the 0 to 10 range of the probability distribution, historical EMR information is used to determine the incidence for all patients that had probability score between 0-10 for a given hospital, clinician, or region. The same operations are then performed for each of the remaining quintiles: 20 to 30 range, 30 to 40 range, 40 to 50 range, and 50 to 60 range and on until 90 to 100 range.

At 830, the normalized patient data is analyzed in order to create “buckets,” or groups, of patients using the model. The buckets of patients are created based on the probability predictions for complications generated by the model that likely lead to readmission, for example, as set forth in FIG. 9. The number of buckets to be created may vary from 2 to N, where N may be different for different types of medical procedures to be performed and/or their associated risks.

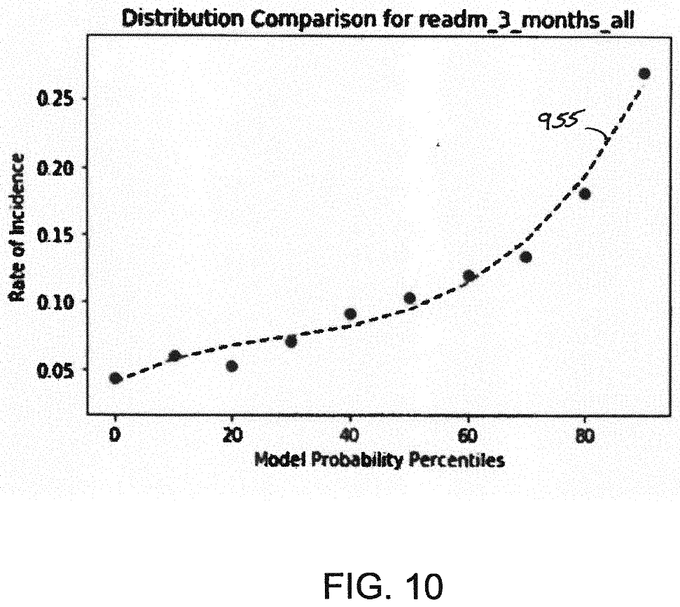

At 840, the rate of incidence (e.g., actual rate of readmission) for the patients within each bucket may be determined for each patient and for each complication and for a given institution. The patients classified into the same bucket may all have a similar probability of complications. FIG. 10 illustrates an example of a graph where normalized patient data is classified into ten buckets relating to readmission after three months from a knee replacement surgery. Each point on the graph indicates a corresponding rate of incidence for a respective one of the buckets. The dotted line 955 is a trend line following the points.

In one embodiment, the accuracy of the predictive model generated for each risk outcome and/or each procedure may be tested using the following metrics: model accuracy, sensitivity, specificity, precision, recall, Kappa Statistics, and F-1 score. The recall and precision of different tools may be evaluated using, for example, Receiver Operating Characteristics (ROC) curves and Area Under the Curve (AUC) analysis. Various leaning methods may also be employed. The following table shows examples of these values computed for the ROC curve corresponding to FIG. 5, as previously discussed.

| | precision | recall | f1-score | support |
|---|---|---|---|---|
| 0 | 0.95 | 0.67 | 0.78 | 2985 |
| 1 | 0.17 | 0.65 | 0.27 | 314 |
| micro avg | 0.66 | 0.66 | 0.66 | 3299 |
| macro avg | 0.56 | 0.66 | 0.53 | 3299 |
| weighted avg | 0.87 | 0.66 | 0.73 | 3299 |

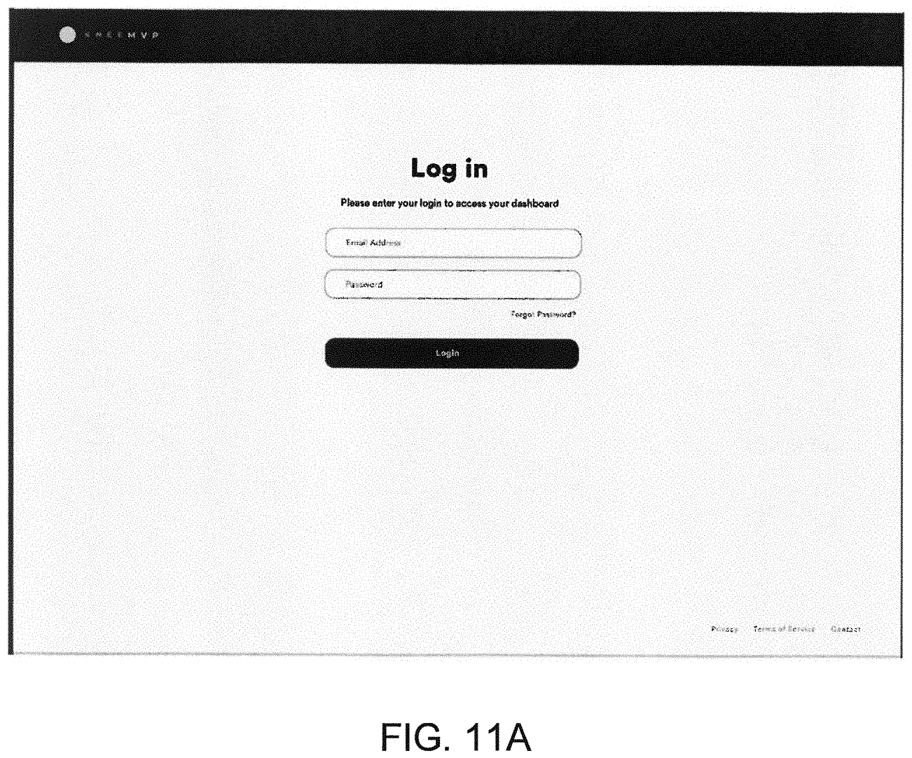

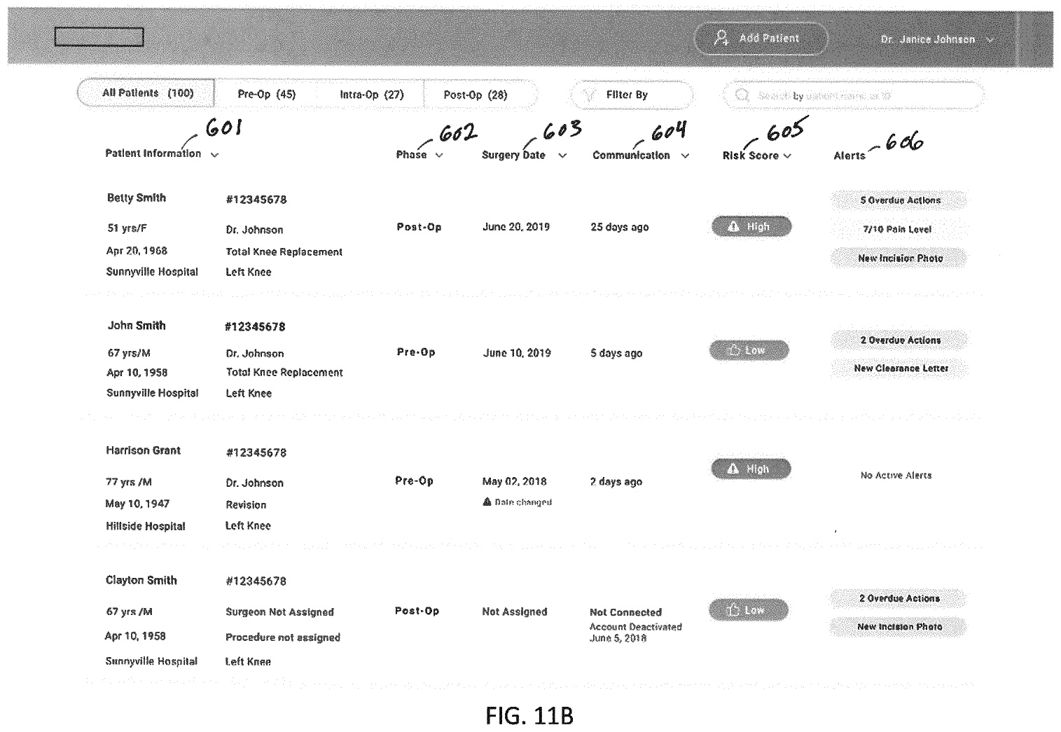

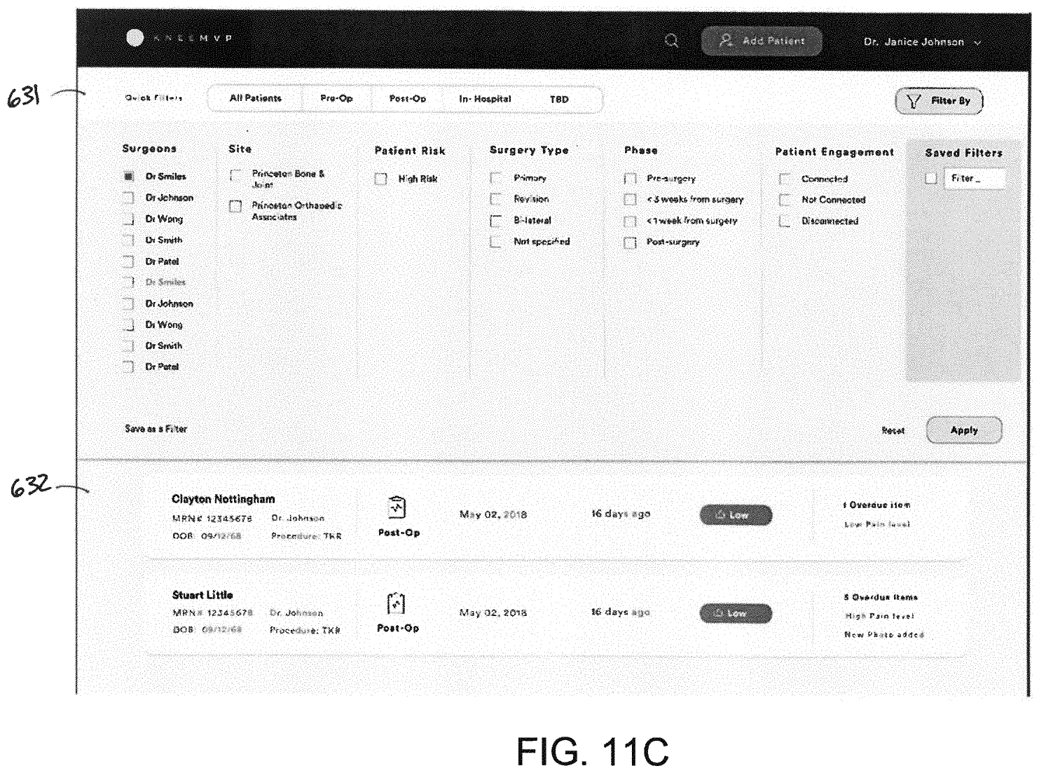

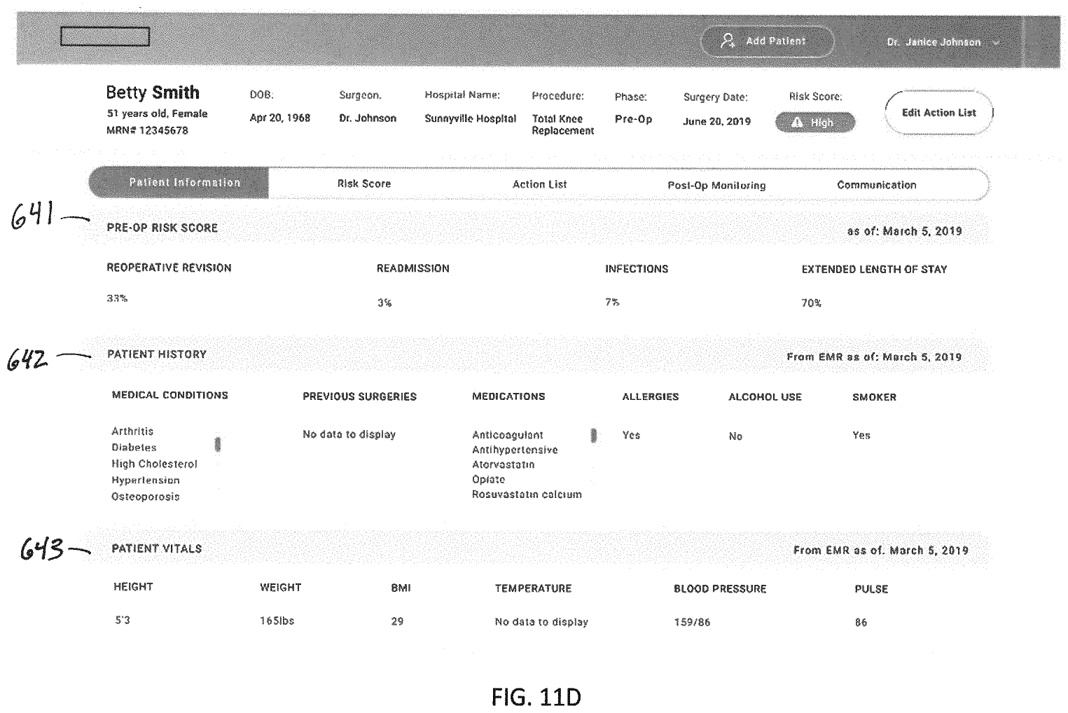

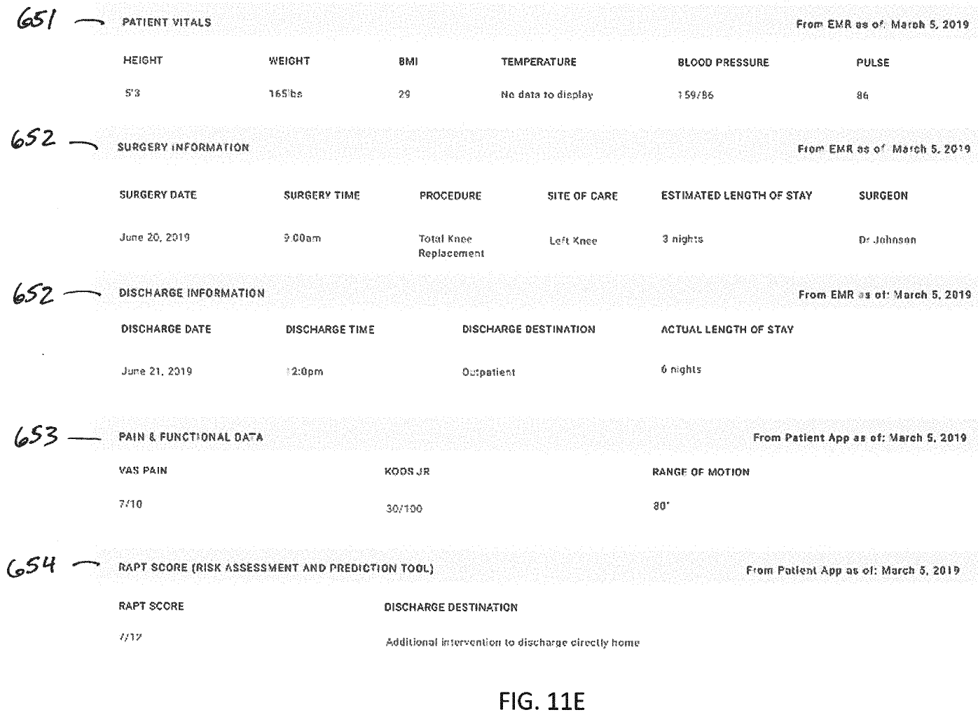

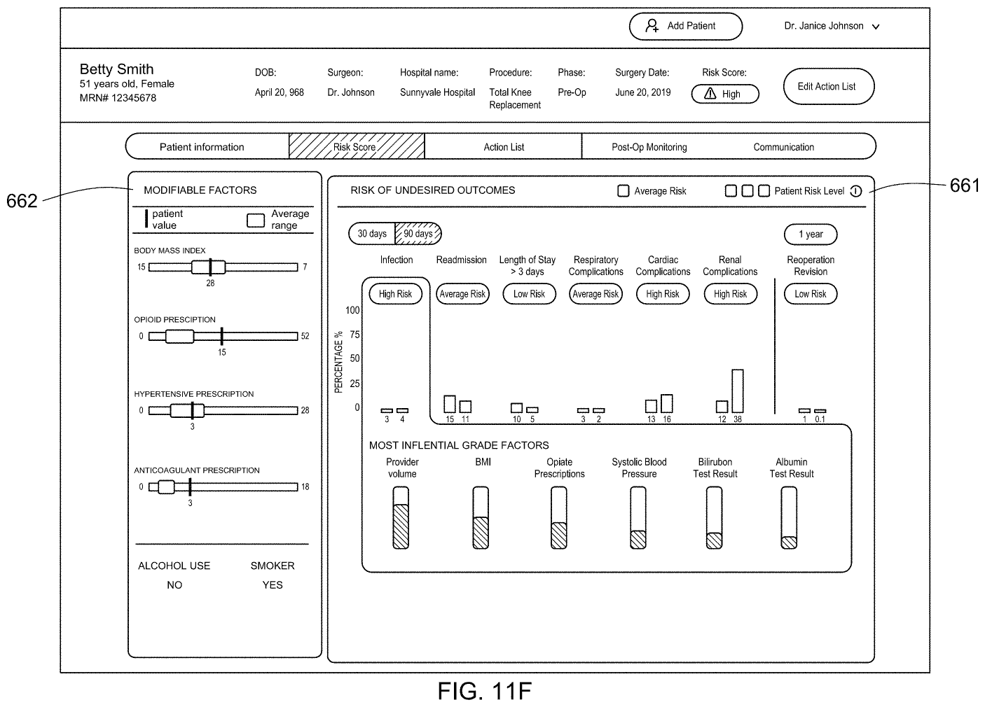

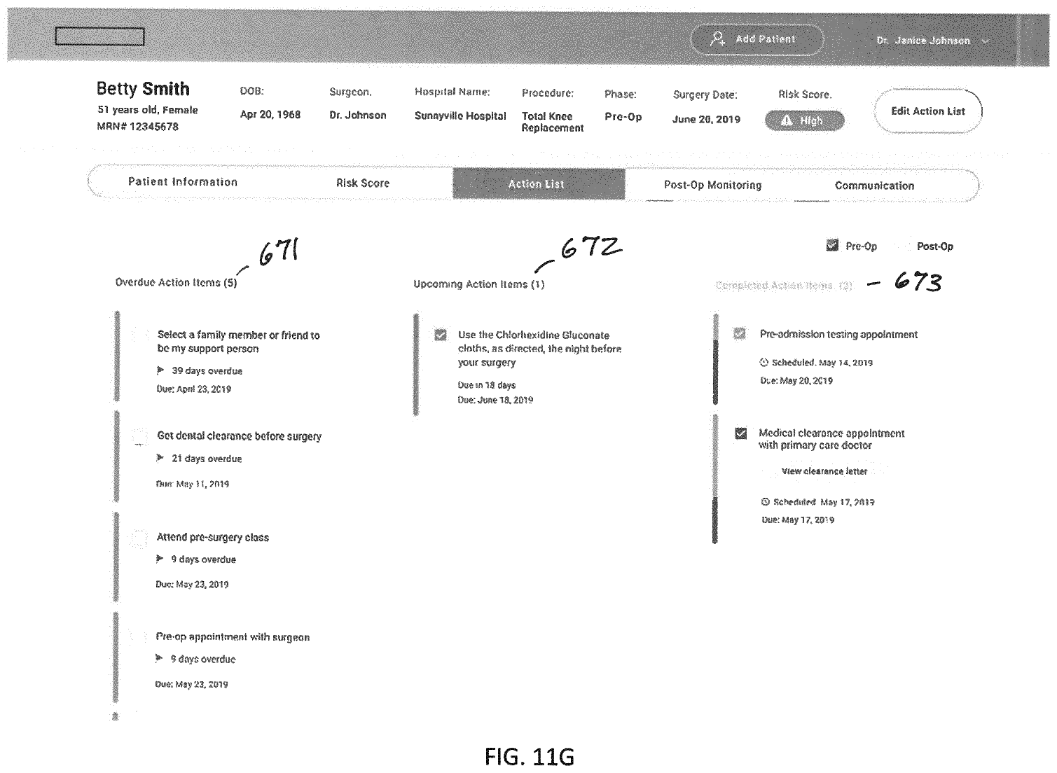

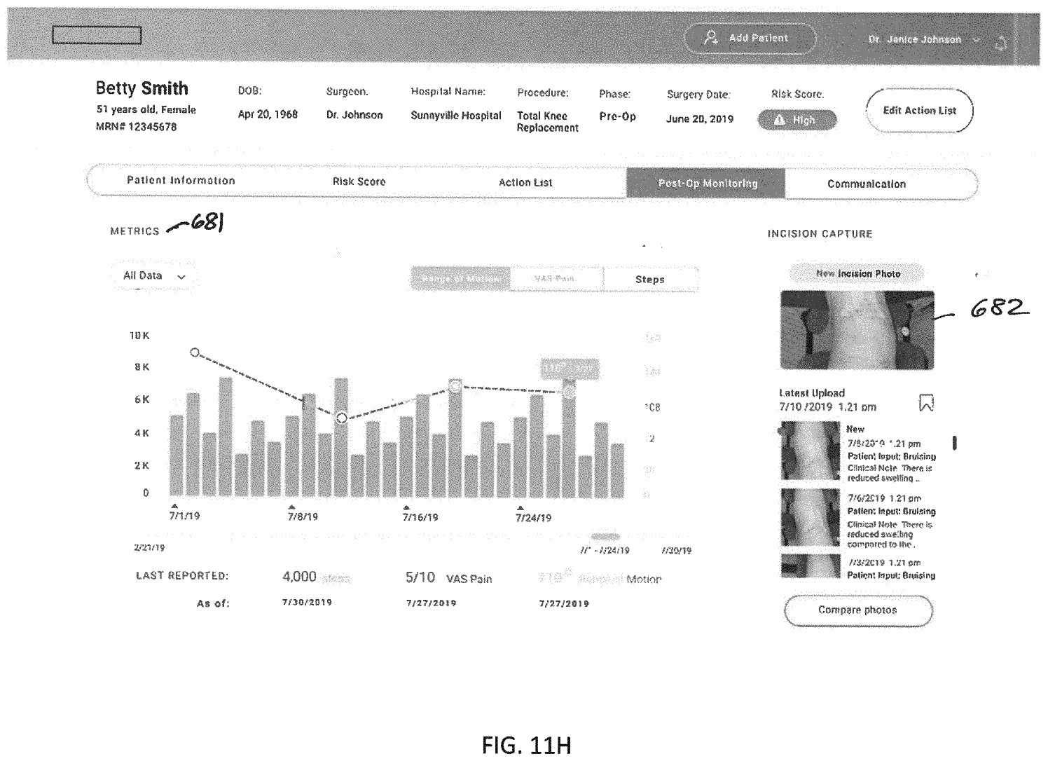

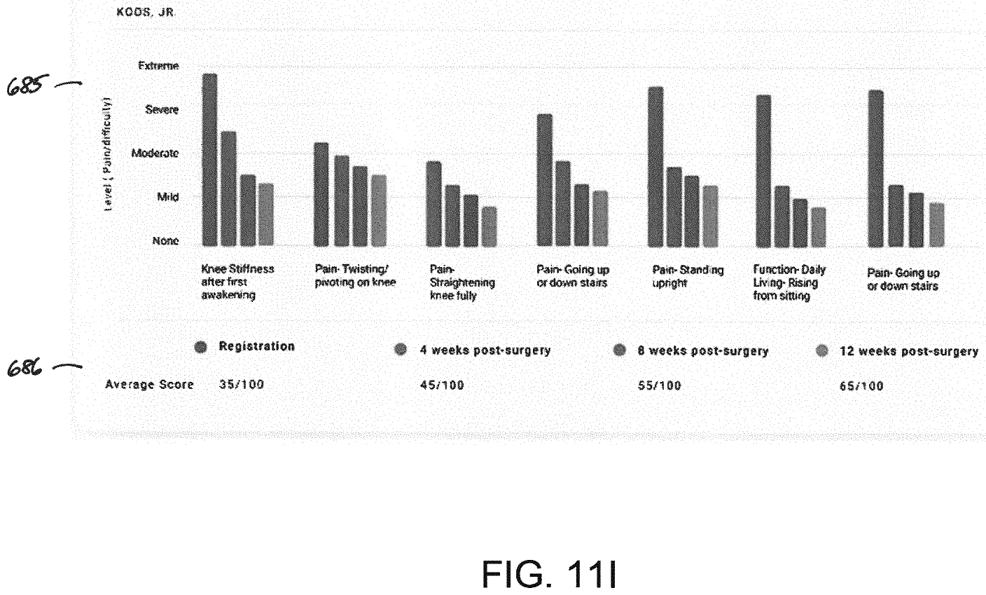

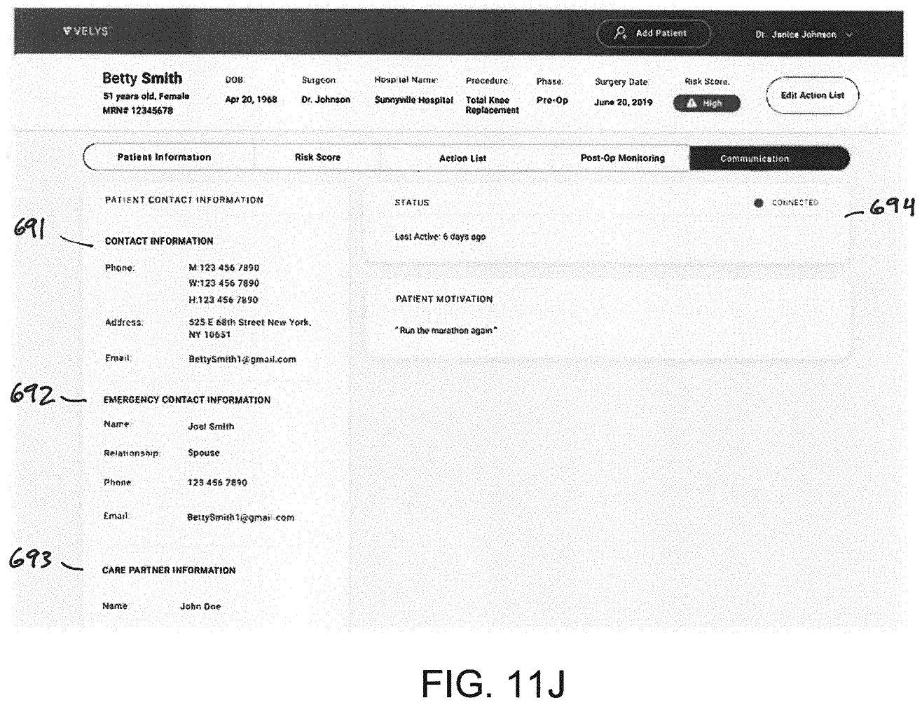

FIGS. 11A to 11I illustrates examples of additional screens that may be generated by the system for predicting patient risk outcomes for various types of medical procedures and/or treatment options. FIG. 11A illustrates a log in screen which may be accessed, for example, by a remotely located device equipped with an application or program that performs the operations of the predictive modeling engine 100 and that displays the screens discussed herein that are generated based on those operations and/or additional interactive controls and information. As previously indicated, the remotely located device may be a mobile device or may be a computer connected to a server or computer running software of the predictive modeling engine.

FIG. 11B illustrates a screen including a list of patients of a particular doctor (Dr. Johnson). The list of patients 601 is displayed with the procedure to be performed, personal information (e.g., date of birth), insurance number, phase of treatment (e.g., pre-operative, post-operative, etc.) 602, the date of the surgery or treatment to be performed 603, the last communication with the patient 604 and/or the number of days left until the surgery or treatment, and risk score 605 generated by the predictive modeling engine. The risk score may be indicated with alerts 606 or special features to distinguish, for example, whether the risk outcome calculated by the predictive modeling engine determined the upcoming procedure or treatment to be of high or low risk. This is an overall risk score where the logic will be determined by the hospital or clinician. For example, the clinician may prefer that all patients with readmission greater than 5% should be classified as high risk patient or someone with 3 risk scores greater than average patient. Additional information may also be displayed, such as alerts. The alerts may indicate the pain level the patient is currently experiencing, whether there are any overdue items that need to be attended to prior to the treatment or surgery (e.g., insurance-related pre-authorizations, pre-treatment preparations or procedures, etc.). The alert may be coming from a patient app that interfaces with the patient. This app will allow the patient to enter pain and functional scores, track their progress toward preparedness for surgery or track post-op progress. The screen of FIG. 11B may further include a filter for filtering the patients of Dr. Johnson by date, name, treatment, and/or one or more other selected parameters.

FIG. 11C illustrates a screen which includes an interface for searching patient-related information for a given hospital where a hospital personnel (i.e. nurse, care navigator, physician assistant) keeps track of patients for more than one surgeon or clinician. The screen includes a filter section 631 and a results section 632. The filter section 631 includes a plurality of options which may be selected by a user for performing a custom search. The options include selections for defining the scope of the search. For example, the scope of the search may be defined by selecting all patients, pre-operative patients, post-operative patients, and in-hospital patients. The options may also include searching by surgeon name, site of the treatment or surgery, patient risk (e.g., high, low, etc.), type of surgery, phase of surgery (e.g., pre-operative, post-operative, etc.), and patient engagement. By selecting one or more of these options, custom searches may be performed that are filtered to the specific preferences of the healthcare professional (e.g., doctor, staff, clinician, administrative personnel, etc.).

The results selection 632 shows results of the search performed based on the options selected in the filter section 631. The results may include the name of the patient, patient ID or medical insurance number, doctor name, type of procedure. The results may also include the status or phase of the patient (e.g., post-operative, pre-operative, etc.), date of the treatment or surgery, the number of days to go until the treatment or surgery, and an indication of the level of risk associated with one or more of the risk outcomes computed by the predictive modeling engine 100. The results may also provide an indication of the pain level the patient is currently experiencing (or as last recorded) and any items that are overdue in relation to the patient and the treatment or surgery he has undergone or will undergo.

FIG. 11D illustrates a screen which summarizes medical information for a patient, which includes a score 641 computed by the predictive modeling engine 100 for one or more risk outcomes relating to a treatment or surgery. The screen may include a summary section from an EMR including the name, age, sex, identification number or medical insurance number, and other statistics, e.g., date of birth, surgeon name, procedure (e.g., treatment surgery, etc.), and phase.

The goal of this screen is to summarize the patient information from an EMR to enable the clinician to quickly assess the health of the patient and candidacy for a given procedure. A screen that has all this information save the time that it would have taken a clinician to find this information in the EMR. The screen may also include selectable tabs for filtering the information to be presented. The tabs may include, for example, patient profile information, risk score(s) computed by the predictive modeling engine 100 for corresponding risk outcomes, progress made to surgery, progress made toward a defined goal, and communication information.

In the example shown in the screen of FIG. 11D, the profile information tab has been selected. When this tab is selected, a pre-op risk score section 641, a patient history section 642, and a patient vitals section 643 is shown. The pre-op risk score section 641 may provide an indication of one or more risk outcomes having the greatest probabilities of occurring given the type of treatment or surgery to be performed for the patient. In this example, the predictive modeling engine 100 has determined that there is a 33% chance that patient Betty Smith will experience re-operative revision, a significant chance that this patient will have to be readmitted, a 7% chance that this patient will experience an infection, and a 70% chance of an extended length of stay. The patient history section 642 may include information on the medical conditions, previous surgeries, medications, allergies, and social factors (smoker, driver, recreational drug user, etc.) of patient Betty Smith. The patient vitals section 643 may indicate height, weight, BMI, temperature, blood pressure, and pulse.

FIG. 11E illustrates a screen (if you scroll down on the patient information tab) including a patient vitals section 651, a surgery & discharge information section 652, a pain & functional data section 653, and a RAPT score section 654. The surgery & discharge information section 652 may include information indicating surgery date, time, type, estimated length of stay, medical facility (site), and surgeon. Section 652 may also include information indicating discharge date, time, destination, and actual length of stay. The pain & functional data section 653 may include information indicating VAS pain, KOOS JR information, and range of motion. The information in this section may be different for different procedures. The RAPT score section 654 provides an indication on the most likely discharge site of care (i.e. home, home health, skilled nursing facility).

FIG. 11F illustrates a screen that is displayed when, for example, the risk score tab in FIG. 11D is selected by a user. This screen includes a screen similar to the screen of FIG. 3. For example, the screen of FIG. 11F includes a risk outcomes section 661 and a modifiable factors section 662. The risk outcomes section 661 includes a listing of risk outcomes computed by the predicative modeling engine 100, along with textual and graphical indications of the scores computed for each risk outcome. These scores may be displayed in comparison to some benchmark, e.g., average risk score for a given patient population, medical facility, geographical region, etc. The scores may be displayed, for example, based on different selectable time periods, e.g., 30-day, 90-day, and 1 year periods. The scores for each risk outcome may change for different time periods, for example, given patient data and condition and type of procedure (treatment, surgery, etc.) to be performed. Each of the risk outcomes in section 661 may be selectable. When each of the risk outcomes is selected, the risk outcomes section 661 may display a corresponding listing of most influential modifiable factors relating to the selected outcome.

The modifiable factors section 662 provides an indication of the one or more factors relating to the patient under consideration that may have an effect on the risk outcomes. In the example shown, the modifiable factors include body mass index of the patient, whether the patient has an opioid prescription, hypertensive prescription, or anticoagulant prescription, and whether the patient is a smoker or alcohol drinker. Values for each of the factors may be set or change, for example, along a sliding scale, by a user. Changes to these factors may produce corresponding changes to the risk outcome scores, as computed by the predictive modeling engine 100. As the factors of the patient change, a healthcare professional may therefore be able to determine what affect such a change may have on the risk outcome given the procedure to be performed.

FIG. 11G illustrates a screen which may be displayed, for example, when the progress to surgery tab in FIG. 11D is selected. This screen includes information indicating a care plan for the patient including overdue items 671 that are required (e.g., either before or after the procedure is performed), upcoming action items 672 to be performed, and completed action items 673.

FIG. 11H illustrates a screen which may be displayed, for example, when the post-operative monitoring tab in FIG. 11D is selected. This screen includes information indicating a patient goal to be achieved after the procedure and various predetermined metrics 681 relating to the patient and the procedure that will be or has been performed. This screen may also include a photo 682 of an incision captured by the patient and sent through to the health care professional to assess healing of a wound site. The images may show, for example, the incision made during surgery for the patient and/or any areas of concern relating to the incision, e.g., swelling, infection, etc. The images may show, for example, patient condition during different stages of the treatment or surgery. This section may be expanded to show additional images that may be stored in a database, including clearance photos, gait, range of motion, mobility.

FIG. 11I illustrates additional information that may be included in or access from the screen in FIG. 11H. The screen in FIG. 11I may include a section 685 indicating different levels of pain experienced for different motions, movements, or other aspects relating to the area operated on or activities the patient may have difficulty with as a result of the operation. A section 686 indicating a corresponding score may be associated with the bar graph for each pain/difficulty categories.

FIG. 11J illustrates a screen which may be displayed, for example, when the communications tab in FIG. 11D is selected. The screen may include information indicating contact information for the patient 691, emergency contact information for the patient 692, care partner information 693, and status of the patient 694.

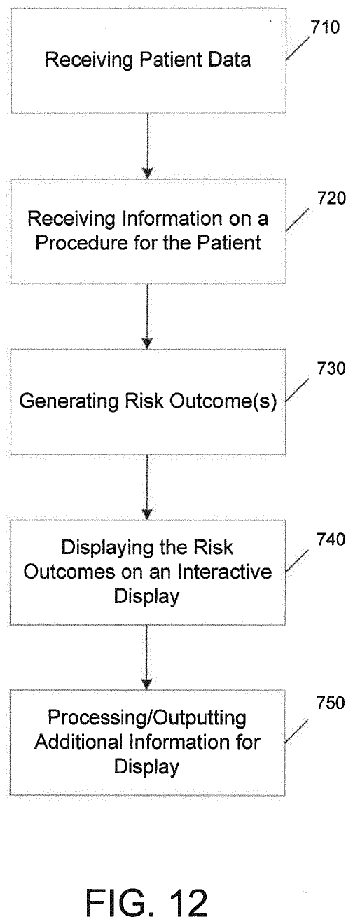

FIG. 12 illustrates an embodiment of a method for predicting patient risk outcomes for various types of medical procedures (e.g., surgeries, treatments, etc.). The method includes, at 710, receiving patient data from one or more databases. The patient data may include information derived from electronic medical records and/or any of the other information obtained from data sources 10 to 50 in FIG. 1. At 720, information is obtained on a type of medical procedure to be performed for the patient. At 730, the predictive modeling engine 100 generates one or more risk outcomes for the patient based on the procedure and the patient data. The risk outcomes may include risk scores computed, for example, by a machine-learning algorithm or another algorithm. At 740, a signal is received to display the risk outcomes (including the risk scores) on an interactive display. At 750, additional signals may be received from a user for generating additional screens of information as described herein.

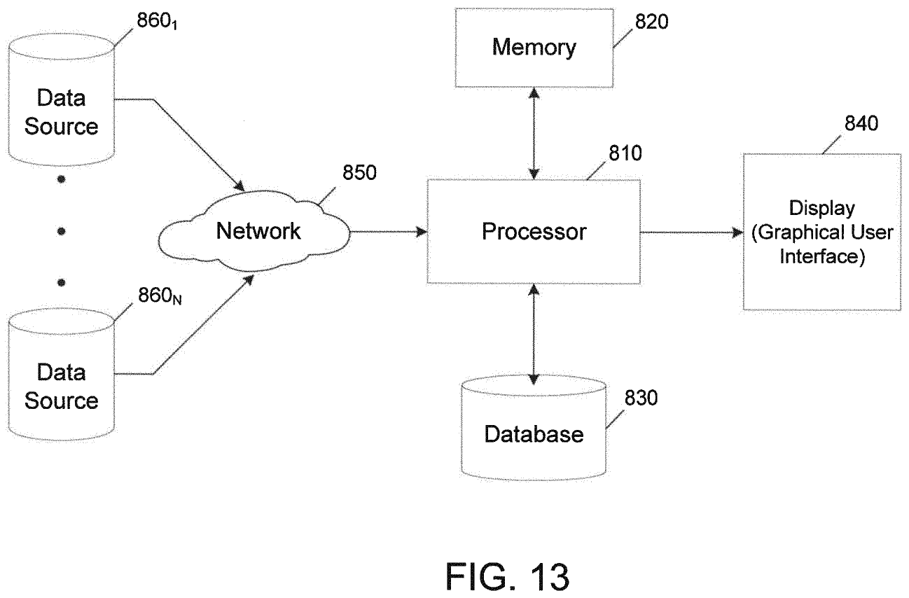

FIG. 13 illustrates an embodiment of a processing system that may be used to implement the embodiments described herein. The processing system includes a processor 810, a memory 820, a database 830, and a display 840. The processor may be any type of logic implemented in hardware, software, or both, for executing programs and instructions stored in the memory 820. The programs and instructions may include one or more of the algorithms which cause the processor 810 to perform operations of the system and method embodiments. For example, the one or more algorithms may correspond to the predictive modeling engine 100 of FIG. 1, which, for example, may be machine-learning and linear regression algorithms. The memory 820 may any one of a variety of non-transitory computer-readable mediums storing the aforementioned programs and/or instructions. The memory 820 may also store instructions for generating the graphical user interface (and its associated screens as described herein) on display 840. The database 830 may store various forms of information to be used by the processor in executing the predictive modeling engine and/or the graphical user interface. The processor may perform these operations based on information stored in one or more databases, 860₁ to 860_{N}, which may be coupled to the processor 810 through one or more networks 850.

### Technological Innovation

The embodiments described herein provide a useful tool for helping physicians, specialists, nurses, and other healthcare professionals understand the risks associated with performing a specific procedure (e.g., treatment, surgery, etc.) on a patient, given that the particular condition and circumstances relating to that patient. These embodiments compute various risk assessments using a predicative modeling engine that employs, for example, one or more machine-learning algorithms to determine and score the likely risk outcomes associated with the procedure to be performed. The algorithms may be trained, for example, using test and actual sets of data, and the risk outcomes scored by the algorithms may improve with accuracy based on feedback and the number of patient cases.

In addition, a graphical user interface may generate interactive screens that present the risk outcomes in association with other information that may provide a comprehensive indication of the risk outcomes and modifiable factors that may affect those outcomes. The interactive screens may also service as a convenient and efficient tool for use by surgeons and other professionals in making the decision whether to perform procedures for a given patient, and to then track the condition of that patient once the procedures is performed. This information displayed on the screens may be generated, in whole or part, based on the computations performed by the predicative modeling engine and/or information stored in one or more databases. The graphical user interface may be accessible, for example, through a website or an application downloaded on a user device.

The embodiments described herein may coordinate and communicate information between and among healthcare professionals and/or patients. For the patient, this information may include notification of the surgery date, access to and monitoring of patient care plans, access to educational materials, reminders as to the schedule(s), medication(s), and care for patients, and content relating to caregivers. For healthcare professionals, the information that may be coordinated and communicated by the embodiments described herein may include some of the same information, as well as relate to tracking patient progress and capture deviations and tracking patient usage of education materials.

The embodiments described herein may also help to optimize patient recovery. For the patient, this may involve providing information to help understand the progress of steps and ROM compared to milestones set by the healthcare provider and the exercises for completion. In addition, images and/or other information captured during treatment or surgery may be stored and displayed. The images may include colonoscopy images, incision images, and/or other images that may provide an indication of patient condition, results of surgery, and/or risks that the patient may face after the surgery or treatment is performed. For the healthcare provider, the graphical user interface may display reminders, scheduling, communication information (e.g., through an app or the internet), medications, and care information. The embodiments may also allow content to be shared with other healthcare professionals, including doctors, nurses, specialists, and caregivers.

The graphical user interface may also display screens indicating pain, satisfaction, KOOS-JR, and PROMIS-10 information, screens that track patient progress and ROM (pre- and post-surgery), patient medical adherence (post-op), and patient adherence to physical therapy plans.

The graphical user interface may also display screens indicating patient stratification information. For the patient, this information may help the patient understand the risks associated with the procedure, the length of stay at the hospital, rehab center, or other medical facility that is expected if the procedure is performed, whether or not the patient is a good candidate for the procedure. For the healthcare provider, the information may indicate which patients are considered to be high risk patients for a given surgery, the likelihood that various risk outcomes will occur, and the discharge site of care based on RAPT or hospital practice. The information may also facilitate a discussion of the risks of surgery, that may be used as a basis for setting patient expectations and enable shared decision-making for surgery. Additional information may include outlining the factors that drive the risk of undesired outcomes, plans to optimize patient success pre-operatively, and educational content indicating factors that may reduce the risk outcomes, e.g., cutting out smoking and drinking.

The methods, processes, and/or operations described herein may be performed by code or instructions to be executed by a computer, processor, controller, or other signal processing device. The code or instructions may be stored in the non-transitory computer-readable medium as previously described in accordance with one or more embodiments. Because the algorithms that form the basis of the methods (or operations of the computer, processor, controller, or other signal processing device) are described in detail, the code or instructions for implementing the operations of the method embodiments may transform the computer, processor, controller, cloud computing or other signal processing device into a special-purpose processor for performing the methods herein.

The processors, engines, and other signal generating and signal processing features of the embodiments disclosed herein may be implemented in logic which, for example, may include hardware, software, or both. When implemented at least partially in hardware, the processors, engines, and other signal generating and signal processing features of the embodiments may be, for example, any one of a variety of integrated circuits including but not limited to an application-specific integrated circuit, a field-programmable gate array, a combination of logic gates, a system-on-chip, a microprocessor, or another type of processing or control circuit.

When implemented in at least partially in software, the processors, engines, and other signal generating and signal processing features of the embodiments may include, for example, a memory or other storage device for storing code or instructions to be executed, for example, by a computer, processor, microprocessor, controller, or other signal processing device. The computer, processor, microprocessor, controller, or other signal processing device may be those described herein or one in addition to the elements described herein. Because the algorithms that form the basis of the methods (or operations of the computer, processor, microprocessor, controller, or other signal processing device) are described in detail, the code or instructions for implementing the operations of the method embodiments may transform the computer, processor, controller, or other signal processing device into a special-purpose processor for performing the methods described herein.

Although the various example embodiments have been described in detail with particular reference to certain exemplary aspects thereof, it should be understood that the invention is capable of other example embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

1. A system for processing information, comprising:
an input configured to receive data relating to a patient through a network;
a display controller configured to control output of information on a display; and
a predictive modeling engine configured to:
compute one or more risk outcomes based on the patient data, the one or more risk outcomes computed for a procedure to be performed on the patient, each of the one or more risk outcomes including a risk score for the patient;
normalize the patient data in quintiles, and
generate buckets of patients for the normalized patient data.
wherein the risk outcomes are determined based on the buckets of patients,
wherein the display controller is configured to control display of information indicative of the risk score for the patient and one or more modifiable factors, the predictive modeling engine configured to change the risk score based on changes in the one or more modifiable factors.

2. The system of claim 1, wherein the risk score corresponds to a probability of readmission of the patient after the procedure is performed.

3. The system of claim 2, wherein the probability of readmission is based on a likelihood that the patient will experience a complication after the procedure.

4. (canceled)

5. The system of claim 1, wherein the predictive modeling engine is configured to compute the one or more risk outcomes based on lab results, medications, or both.

6. The system of claim 1, wherein the predictive modelling engine is configured to generate a recursive features elimination with cross validation model to compute the one or more risk outcomes.

7. The system of claim 1, wherein the predictive modelling engine is configured to compute the risk score for the patient based on a set of importance values, each of the importance values assigned an importance score.

8. The system of claim 1, wherein the predictive modeling engine is configured to generate a plurality of models for a respective number of medical facilities for the procedure, the models trained based on patient data corresponding to the respective number of medical facilities.
